(19) United States
(12) Patent Application Publication
(10) Pub. No.: US 2008/0029101 A1
(43) Pub. Date: Feb. 7, 2008

(54) Condom and system and method for packaging

(76) Inventors: Max Marvin Abadi, Sante Fe de Bogata (CO); W. Sam Chandoha, New York, NY (US)

Correspondence Address: SCHWEGMAN, LUNDBERG & WOESSNER, P.A., P.O. BOX 2938, MINNEAPOLIS, MN, 55402, US

(21) Appl. No.: 11/497,535

(22) Filed: Aug. 1, 2006

(51) Int. Cl. A61F 6/04

(52) U.S. Cl. 128/844

(57) ABSTRACT

A prophylactic device includes an elongated tubular member that also includes a closed end, and an open end. The prophylactic device also includes a first elongated tab placed substantially parallel to the axis of the elongated tubular member, and a second elongated tab placed substantially parallel to the axis of the elongated tubular member. The open end of the tubular member is rolled toward the closed end and the first elongated tab has a length rolled into the rolled end, and a first free end. The second elongated tab also has a length rolled into the rolled end, and a second free end. The first free end and the second free end are attached to one another to hold the rolled prophylactic device in a non round shape.

### BACKGROUND

Condoms have been a highly favored alternative among men who wish to take part in sexual activities but want to avoid sexually transmitted diseases or avoid pregnancy in their partner. A wide variety of condoms or prophylactic items are constructed in the form of a protective sheath of thin, flexible material. Commonly, condoms are supplied in a rolled up configuration. Most condoms are currently packaged by rolling them and placing them in a plastic wrapper, which helps protect the condom from physical and chemical damage, including that caused by dryness and ultraviolet rays. As packaged, the condom maintains a substantially circular shape. The package is generally square-shaped and includes a substantially circular chamber. The condom is generally flattened, however, the rolled up rim is still the “high point” of the package.

Applying the condom requires opening the package, removing the condom from the chamber, correctly positioning the condom over the penis and unrolling the condom over the length of the penis. In some instances, condoms may be coated with, or otherwise immersed in, one or more compounds providing a lubricant, a spermicide, or a bactericide, all of which render the condom difficult, or at least uncomfortable, to handle during application. In addition, removal may be difficult, or uncomfortable, and may require careful handling to maintain aseptic conditions, or at least a modicum of cleanliness. Use of rolled up condoms in low light conditions, may present some difficulties. Determining a correct initial positioning of the condom is required for correct application. In some instances, an incorrect initial positioning requires the condom to be thrown away. Even if correctly positioned initially, unrolling the condom sometimes does not go smoothly and may lead one to believe the unit was not correctly positioned initially.

### BRIEF DESCRIPTION OF THE DRAWINGS

The invention is pointed out with particularity in the appended claims. However, a more complete understanding of the present invention may be derived by referring to the detailed description when considered in connection with the figures, wherein like reference numbers refer to similar items throughout the figures and:

FIG. 1 is a front view of the card package with a cover giving the appearance of a credit card, according to an example embodiment.

FIG. 2 is a side view of the card package of FIG. 1, showing the thinness of the package, according to an example embodiment.

FIG. 3 is a perspective view of the card package as a user removes one of the adhesive covers over a cavity in the card package, according to an example embodiment.

FIG. 4 is a perspective view of the card package as a user removes a condom from a cavity in the card package, according to an example embodiment.

FIG. 5 is a perspective view of rolled prophylactic removed from the card package, according to an example embodiment.

FIG. 6 is a side view of a prophylactic in an unrolled state and applied to a member, according to another example embodiment.

FIG. 7 is a cut-away side view of rolled prophylactic along line 6-6 of FIG. 5, according to an example embodiment.

FIG. 8 is a side view of a prophylactic 800 in an unrolled state and applied to a member, according to another example embodiment.

FIG. 9 is a top view of partially unrolled prophylactic, according to an example embodiment.

FIG. 10 is a perspective view of a prophylactic with instructions printed on the free ends of the elongated pull tabs, according to an example embodiment.

FIG. 11 is a schematic view of a kit that includes a card package holding at least one condom, individually packaged lubricants and an instruction set, according to an example embodiment.

FIG. 12 is a view of an instruction set associated with the kit of FIG. 9, according to an example embodiment.

FIG. 13 is a flow diagram of a method, according to an example embodiment.

FIG. 14A is a cross sectional view along 14A-14A in FIG. 10, according to an example embodiment.

FIGS. 14A-14E are cross sectional views of various stages of deployment of the prophylactic, according to an example embodiment.

The description set out herein illustrates the various embodiments of the invention and such description is not intended to be construed as limiting in any manner.

### DETAILED DESCRIPTION

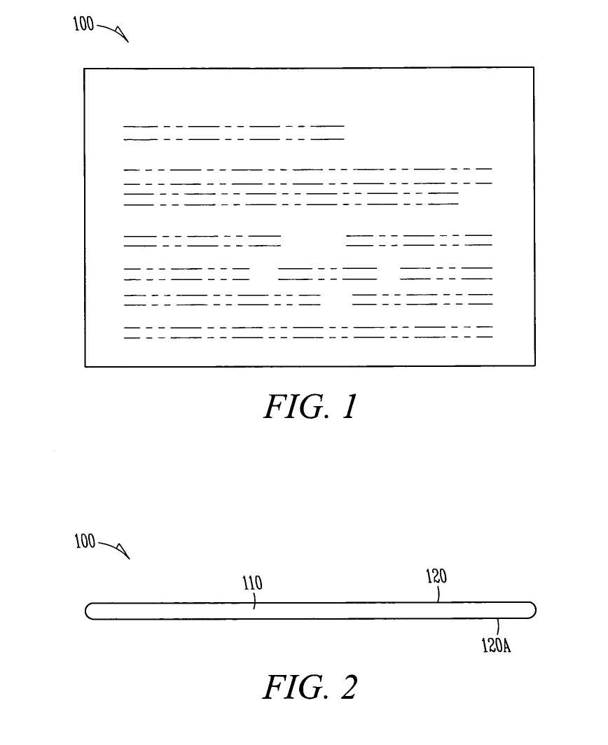

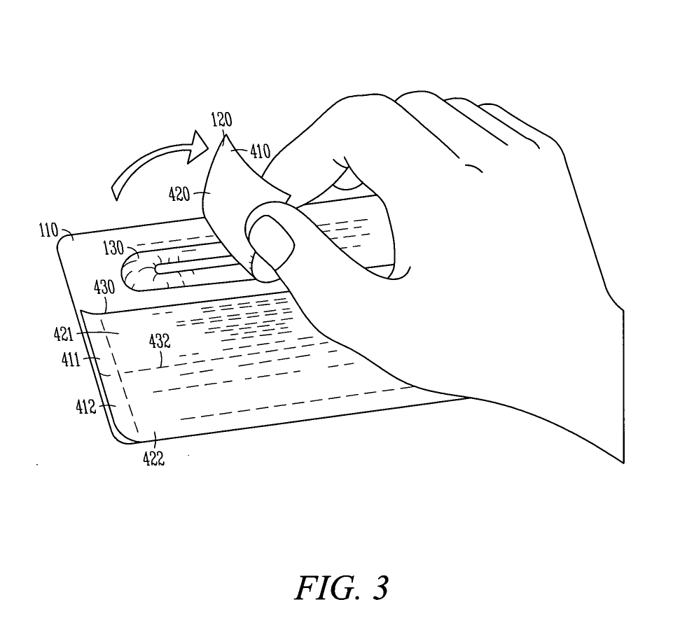

FIGS. 1 and 2 show a card package 100. FIG. 1 is a front view of the card package with a surface having the appearance of a credit card, according to an example embodiment. FIG. 2 is a side view of the card package of FIG. 1, showing the thinness of the card package 100, according to an example embodiment. The card package 100 includes a body 110 and a cover 120. The body 110 is formed from a thin sheet of rigid, but sufficiently plastic, material having properties similar to a credit card. In one embodiment, injectable plastics, preferably PVC or polyethylene, are used in forming the body of the card package 100. The material of the body 110 is cut from a sheet of this material, with dimensions of 8.5 cm by 5.4 cm, similar to those of a credit card. The thickness of the card body 100 is determined by the thickness of a condom which will be housed within the body 100. In one embodiment, the thickness is in a range of 1.8 to 5.0 mm. In another embodiment, the thickness is in a range between 2.0 and 2.5 mm. As mentioned previously, the thickness of the card body 100 is determined by the thickness of the condom that it will house. The card package also includes a cover 120 and 120A. The cover 120 is formed from a sheet of material that is thinner than the card body 110. The sheet that forms the cover 120 and 120A is adhered to the card body 110 with an external adhesive (glue, adhesive, etc.), or a mechanical closure system which may include the use of heat and pressure, ultrasound or any other similar system. It should be noted that the cover 120 can be attached to the card body 110 by other means as well.

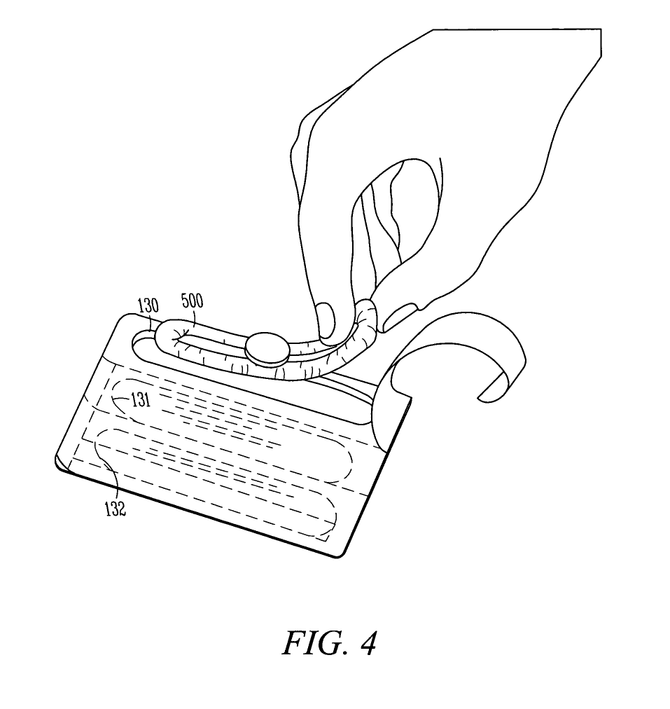

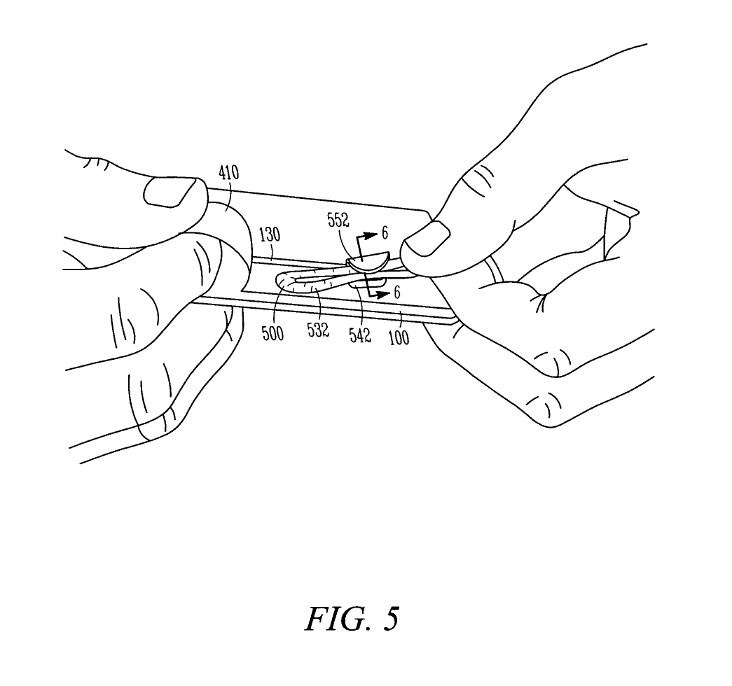

FIG. 3 is a perspective view of the card package as a user removes a portion of the cover 120 from a cavity 130 in the card package 100, according to an example embodiment. FIG. 4 is a perspective view of the card package 100 as a user removes a condom 500 from the cavity 130 in the card package, according to an example embodiment. As shown in FIGS. 3 and 4, the card body 100 includes a cavity 130 therein. The cavity 130 is substantially oblong or oval-shaped. In some example embodiments, there are multiple cavities. FIG. 4 shows an embodiment that includes multiple cavities 130, 131, 132. The cavities 131, 132 are shown in phantom. A condom, such as condom 500, fits within the cavity 130. The condom 500 is in a oval or hour-glass shaped. The cavity 130 or cavities 130, 131, 132 in the card body 110 are formed by using a die which, by pressing the plastic also cuts the plastic, and leaves the cavity 130 or multiple cavities 130, 131, 132. In another embodiment, the package card 100 is formed by injection molding. Injection molding permits a package card 100 to be produced with a cavity or cavities already incorporated. The cavities 130, 131, 132 are sized and shaped to house a condom, such as condom 500. Of course, the size and shape of the cavities 130, 131, 132 can vary depending on the size and shape of the condom, such as condom 500, which are housed within the cavities 130, 131, 132.

The number of cavities can also vary depending on the number of condoms which are to be included in the package card 100. In one example embodiment, the package card 100 has a single cavity 130. In another example embodiment, the package card 100 has a plurality of cavities 130, 131, 132. As shown, the cover 120 can include perforations 430, 432. The perforations provide for a weak line along which the cover can tear away. The perforations 430, 432 separate the cover 120 into separate cover portions 420, 421, 422. Cover portion 420 fits over and seals cavity 130. Cover portion 421 fits over and seals cavity 131. Cover portion 422 fits over and seals cavity 132. As shown in FIGS. 3 and 4, the cover portions 420, 421 and 422 include a tab 410, 411, 412, respectively. The tab 410, 411, 412 is a free end which is free of adhesive and of a size so that it can be lifted and pulled using a person's digits.

During manufacture, the condom 500 is placed in the cavity 130 in the body 110 of the card package 100. In an alternative embodiment, condoms, such as condom 500, are placed in the cavities 430, 431, 432, and other desirable additives are added to the cavities 430, 431, 432. The additives may include ______. The card package 100 is finished by covering it with a very thin sheet or cover 120. The cover 120 is bonded to the body 110 of the card package 100. The cover 120 may be bonded to the body 110 using an external adhesive (glue, adhesive, etc.), or using a mechanical closure system which may include the use of heat and pressure, ultrasound or another similar system. The cover 120 can be pre-perforated with perforations 430, 432 before placing the cover 120 onto the body 110 of the package card 100. Any thin material can be used to form the cover 120, including polyethylene or PVC or similar material. The tabs 410, 411, 412 associated with the cover 120 facilitate the opening of a cavity 130, 131, 132. In one embodiment, the tab is a small corner of the cover 120 or sheet without adhesive associated with each cavity, thereby allowing the user to start from the corner. The cover 120 or sheet may be transparent, but preferably should be printed in order to give the card the external appearance of a credit card (shown as FIGS. 1 and 2). In another embodiment, another similar sheet 120 can also be used to cover the opposite surface of the card package 100 or the front of the card package 100, to give the card a consistent finish. Cover sheet 120A may have printed information about the prophylactic or condom. This information may include the condom's trade name, the condom's characteristics or properties or other related information.

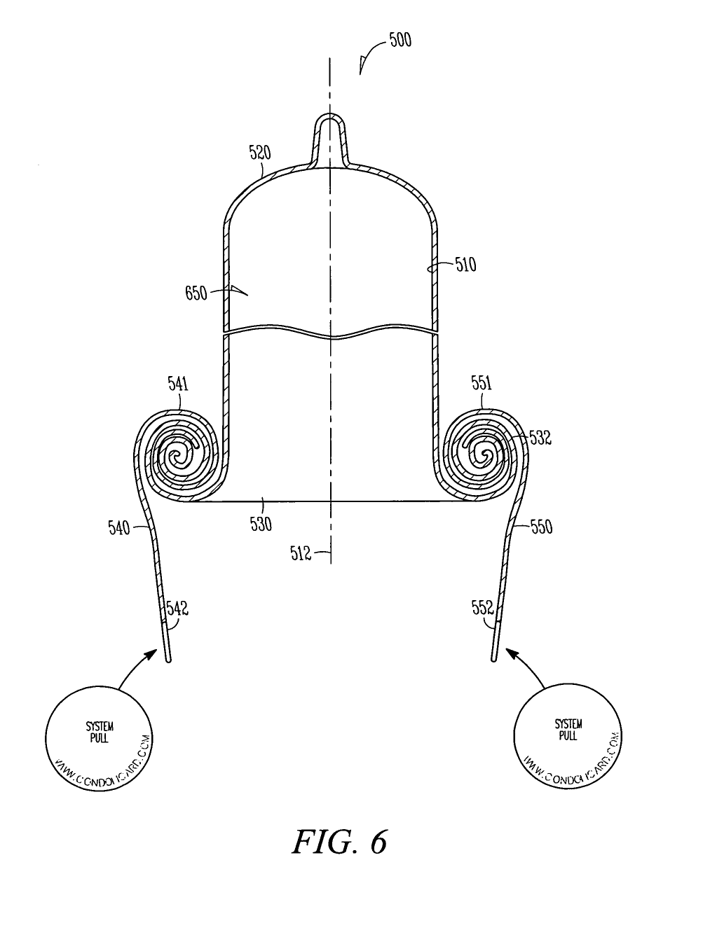

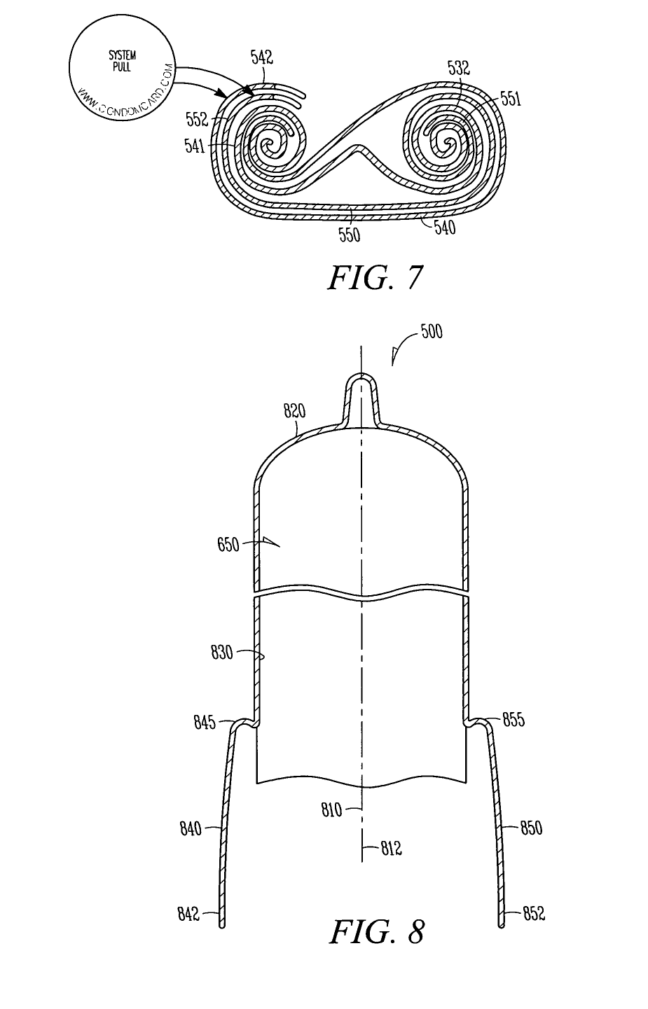

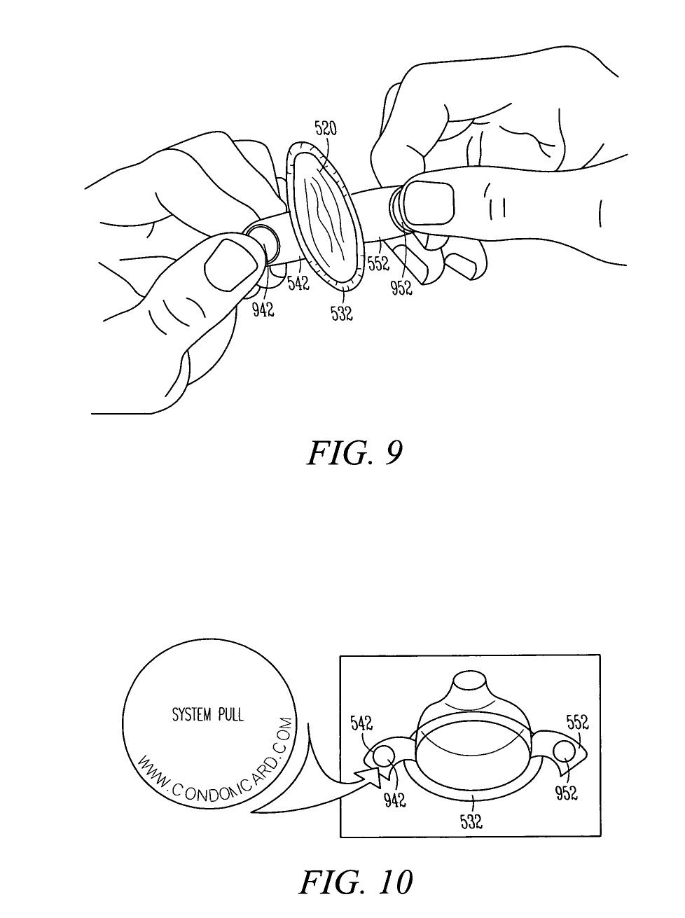

FIG. 5 is a perspective view of rolled prophylactic 500 removed from the card package, according to an example embodiment. FIG. 6 is a side view of a prophylactic 500 applied to a member 650, according to an example embodiment. FIG. 7 is a cut-away side view of rolled prophylactic 500 along line 8-8 of FIG. 5, according to an example embodiment. The prophylactic 500, 700 will now be further detailed while referring to FIGS. 5-7. As shown in FIG. 5, the prophylactic 500 in a rolled up state includes a closed end 520, a rolled end 532 proximate the closed end 520 and a first tab 540 and a second tab 550 with free ends 542, 552, respectively, extending out from the rolled end 532. The free ends 542, 552 are wrapped around the rolled end 532 to hold the rolled end 532 in a non round shape.

Now turning to FIG. 6, the prophylactic 500 is shown in an unrolled state on a member 650. The prophylactic device 500 includes an elongated tubular member 510 that includes a closed end 520, and an open end 530. The prophylactic device 500 also includes a first elongated tab 540 placed substantially parallel to an axis 512 of the elongated tubular member 510, and a second elongated tab 550 placed substantially parallel to the axis 512 of the elongated tubular member 510. The open end 530 of the tubular member 510 is rolled toward the closed end 520 to form a rolled end 532 shown in FIGS. 5 and 7. The first elongated tab 540 has a length 541 rolled into the rolled end 532. The first elongated tab 540 has a first free end 542 that extends beyond the rolled end 532. The second elongated tab 550 also has a length 551 rolled into the rolled end 532. The second elongated tab 550 also has and a second free end 552 that extends beyond the rolled end 532. The first length 541 and the second length 551 are captured in the rolled end 532 (see FIG. 7). The first free end 542 and the second free end 552 are wrapped around the rolled end 532 to hold the rolled prophylactic device in a non round shape (see FIGS. 5 and 7). In one embodiment, the non round shape is substantially elliptical, while in another embodiment, the non round shape is substantially hour-glass shaped. In one embodiment, attaching the first free end 542 of the first tab 540 and the second free end 552 of the second tab 550 includes laying the tabs 540, 550 on one another and wrapping them around the rolled end 532. In another embodiment, releasable adhesives may also be used to attach the first tab 540 and the second tab 550. As shown in FIG. 6, the length 541 and the length 551 of the tabs 540, 550 are not attached to the tubular portion at or near the open end 530 of the prophylactic device 500. In other words, the captured ends merely lay in the rolled portion 532 and are not attached to the main body or the end of the prophylactic device 500.

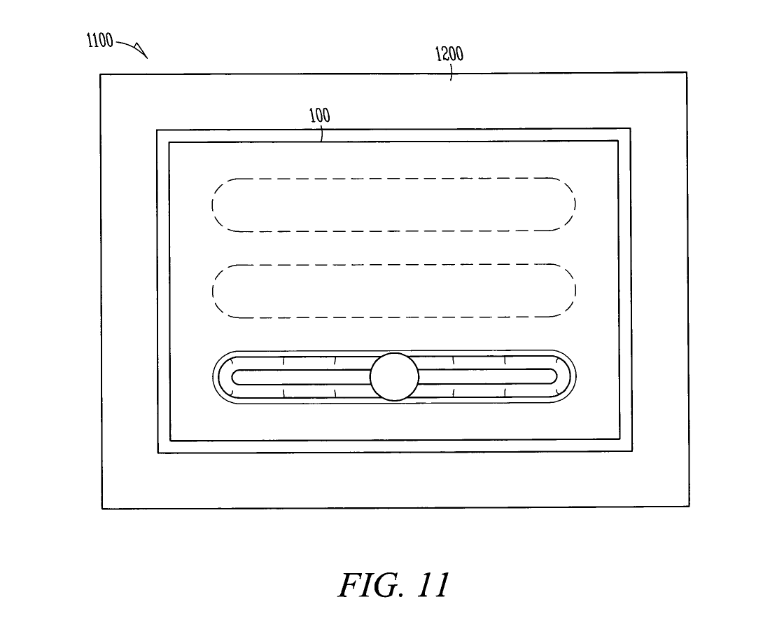

FIG. 8 is a side view of a prophylactic 800 in an unrolled state and applied to a member 650, according to another example embodiment. The prophylactic device 800 is similar to the prophylactic device 500. The prophylactic device 800 includes an elongated tubular member 810 that includes a closed end 820, and an open end 830. The prophylactic device 800 also includes a first elongated tab 840 placed substantially parallel to an axis 812 of the elongated tubular member 810, and a second elongated tab 850 placed substantially parallel to the axis 812 of the elongated tubular member 810. The open end 830 of the tubular member 510 is rolled toward the closed end 820 to form a rolled end, such as rolled end 532 shown in FIGS. 5 and 7. The first elongated tab 840 has a length 841 rolled into the rolled end 832. The first elongated tab 840 has a first free end 842 that extends beyond the rolled end 832. The second elongated tab 850 also has a length 851 rolled into the rolled end 832. The second elongated tab 850 also has and a second free end 852 that extends beyond the rolled end 832. The first length 841 and the second length 851 are captured in the rolled end. The first free end 842 and the second free end 852 are attached to one another and wrapped around the rolled end 832 to hold the rolled prophylactic device in a non round shape (similar to the prophylactic device 500 shown in FIGS. 5 and 7). In one embodiment, the non round shape is substantially elliptical, while in another embodiment, the non round shape is substantially hour-glass shaped. In one embodiment, attaching the first free end 842 of the first tab 840 and the second free end 852 of the second tab 850 includes laying the tabs 840, 850 on one another and wrapping them around the rolled end 832. In another embodiment, releasable adhesives may also be used to attach the first tab 840 and the second tab 850. As shown in FIG. 8, the end of the length 841 and the end of the length 851 of the tabs 840, 850 are attached to the tubular portion at or near the open end 830 of the prophylactic device 800. Having an end of the length 841, 851 of the tabs 840, 850 attached to the main body 810 of the prophylactic device 800 is the main difference between the prophylactic device 800 and the prophylactic device 500. In other words, the example embodiment of the prophylactic device 800, the first elongated tab 840 includes a first attached end 845, and the second elongated tab 850 has a second attached end 855, the first attached end 845 and the second attached end 855 attached to elongated tubular member proximate the open end 830. For the sake of simplicity, the discussion of the remaining figures will refer to the embodiment of prophylactic device 500. It should be understood however, that the most of the features discussed in the remaining FIGs. are equally applicable to either example embodiment of the prophylactic.

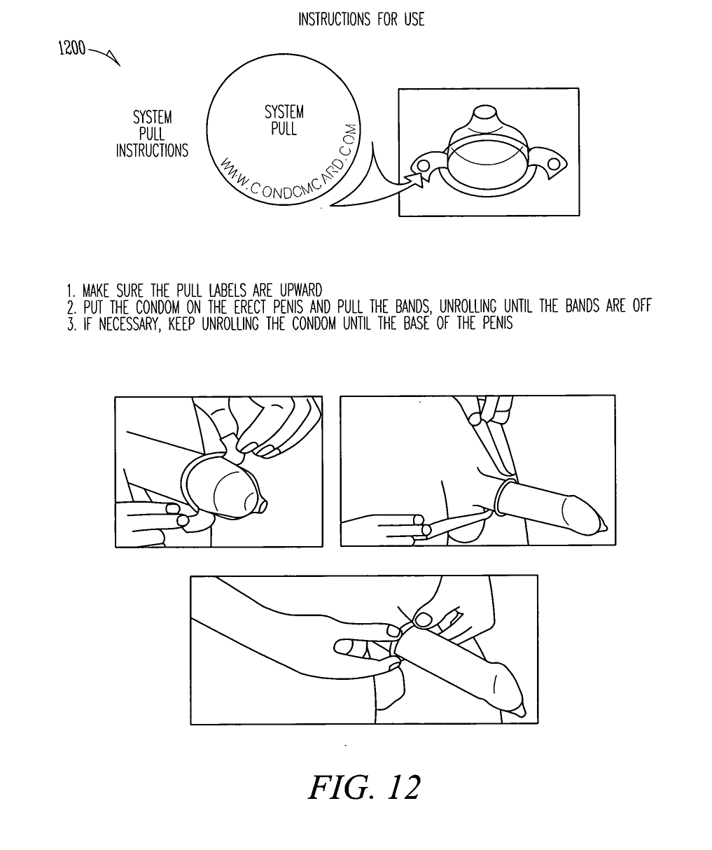

FIG. 9 is a top view of partially unrolled prophylactic 500, according to an example embodiment. As shown in FIG. 9, the first free end 542 and the second free end 552 have been unrolled from their position around the rolled end 532 of the prophylactic device 500. The first free end 542 and the second free end 552 have been separated from one another and the unrolled end 532 is returning to a substantially round shape. As shown in FIG. 9, the first free end 542 includes a first orientation indicator 942, and the second free end 552 includes a second orientation indicator 952. The orientation indicators 942, 952 are viewable from the top when the partially unrolled prophylactic is in a proper orientation for unrolling. In one embodiment, the first orientation indicator 942 and the second orientation indicator 952 include printing. In another embodiment, the first orientation indicator 942 and the second orientation indicator 952 include printed instructions. In still another embodiment, the first orientation indicator 942 and the second orientation indicator 952 are viewable in low light conditions. The orientation indicators 942, 952 viewable in low light conditions could be chemicals that produce light upon mixing or a glow in the dark surface applied to form the low light viewable orientation indicators 942, 952.

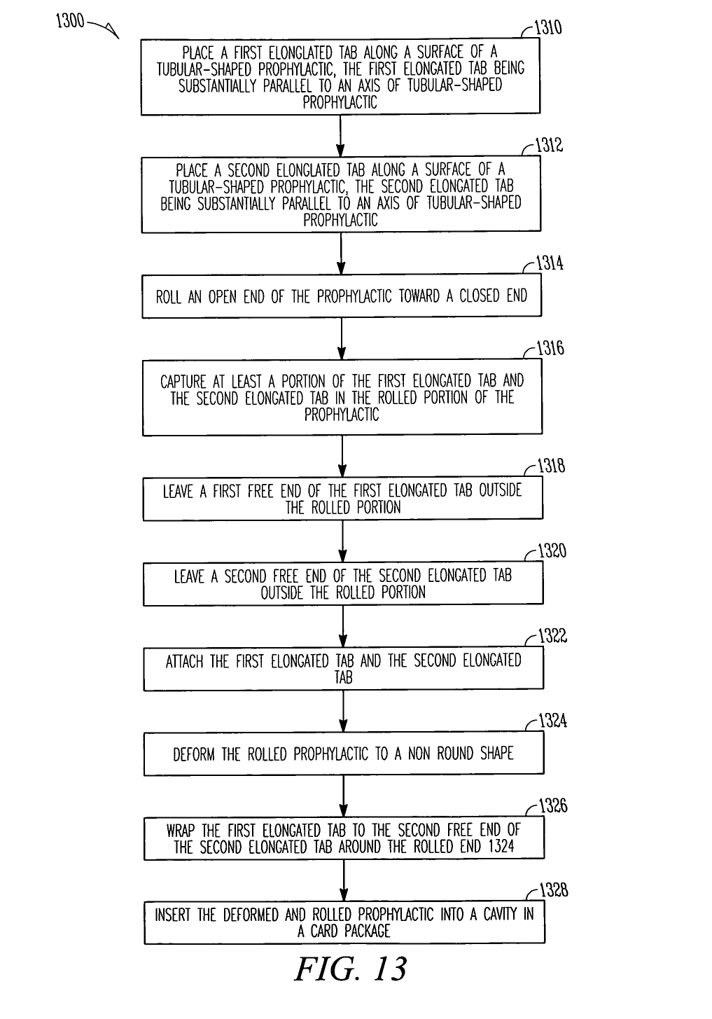

FIG. 10 is a perspective view of a prophylactic device 500 with instructions printed on the free ends 542, 552 of the elongated tabs 540, 550, according to an example embodiment. The instructions printed on the free ends 542, 552 form the first orientation indicator 942 and the second orientation indicator 952. The instructions are viewable when the prophylactic device 500 is in a proper orientation for application or for unrolling.

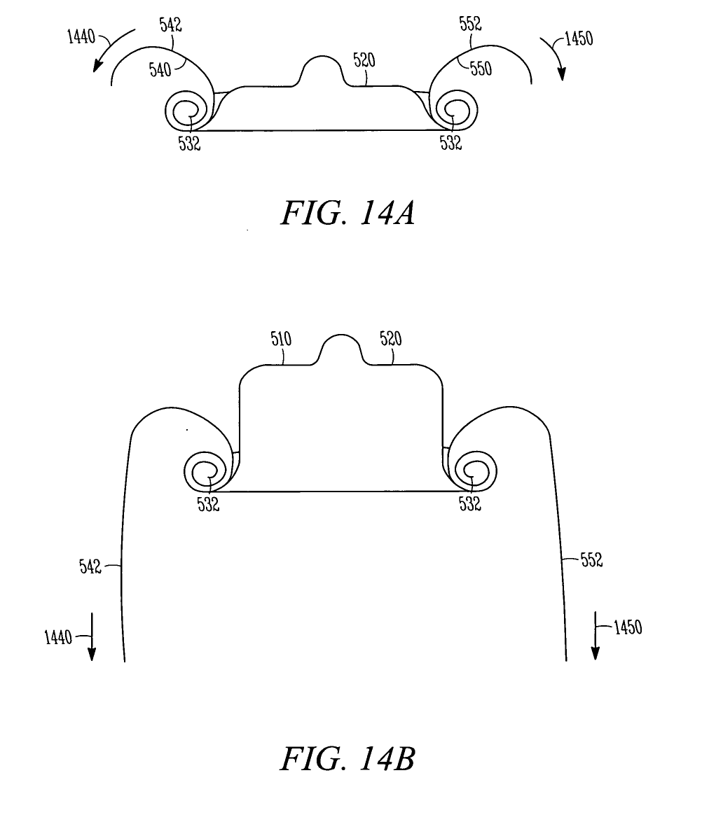

FIG. 11 is a schematic view of a kit that includes a card package holding at least one prophylactic 500 condom, individually packaged lubricants 1110 and an instruction set 1200, according to an example embodiment. The prophylactic device 500 is rolled and placed into a package card. The package card is placed on another card that can be used to carry the instruction set 1200, such as on the back side of the card. The prophylactic device 500 in the rolled state, may also carry some of the instructions associated with the instruction set 1200. Packaged with the card package are individually wrapped portions of a lubricant 1110. In one embodiment, the number of individually wrapped portions of lubricant correspond to the number of individual prophylactic device 500 associated with the package card. The main body of the package card 100 may also carry instructions, in some embodiments. The package card 100 and the lubricant 1110 can be placed in a transparent plastic compartment 1120 attached to the card, in one example embodiment.

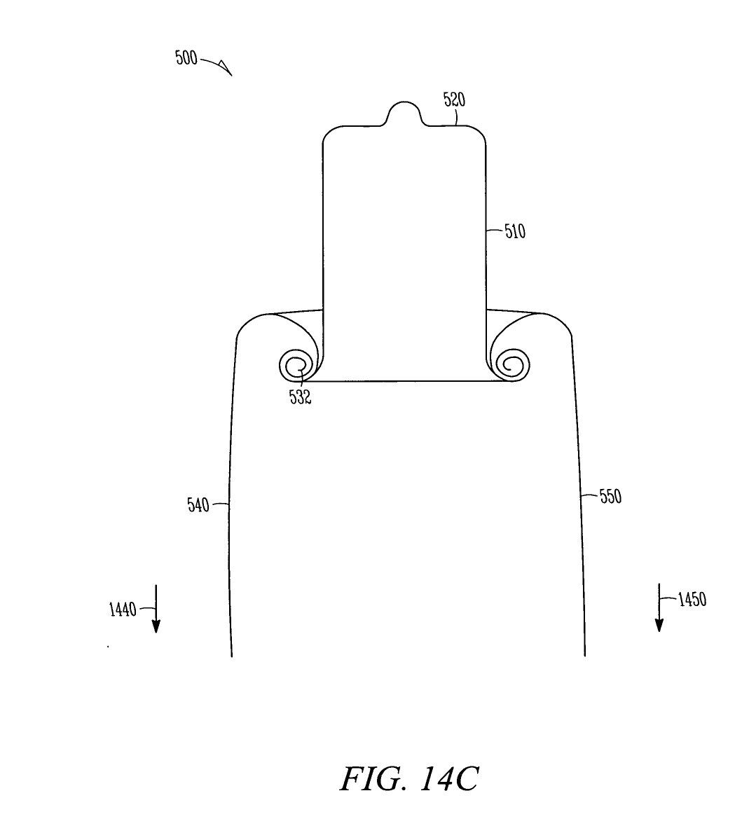

FIG. 12 is a view of an instruction set 1200 associated with the kit of FIG. 11, according to an example embodiment. The instruction set 1200 can include instructions for use of the package cards and the individual prophylactic devices housed within the package card.

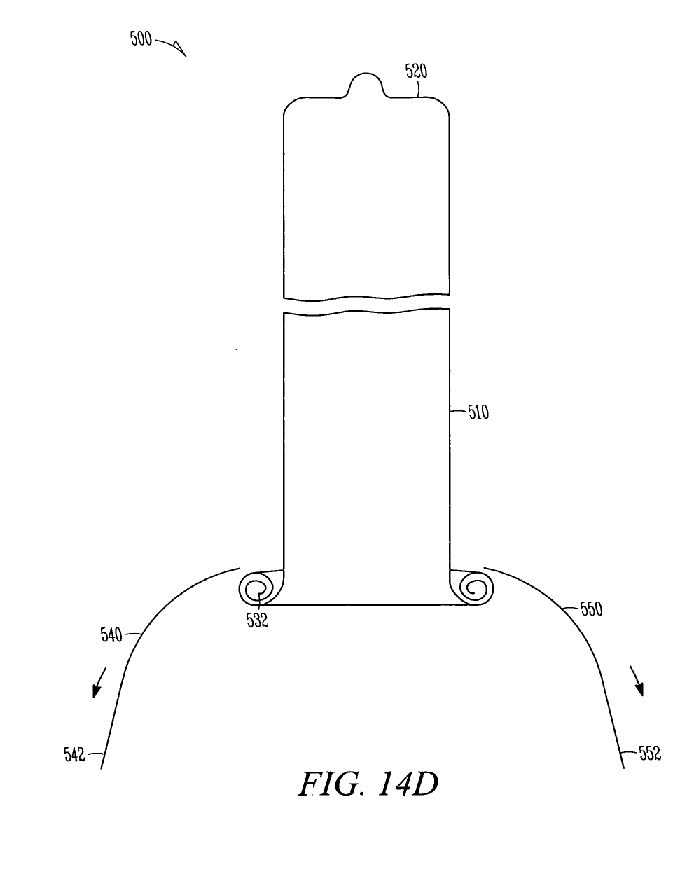

FIG. 13 is a flow diagram of a method 1300, according to an example embodiment. The method 1300 for assembling a card package that includes a prophylactic includes placing a first elongated tab along a surface of a tubular-shaped prophylactic 1310, the first elongated tab being substantially parallel to an axis of tubular-shaped prophylactic, and placing a second elongated tab along a surface of a tubular-shaped prophylactic 1312, the second elongated tab being substantially parallel to an axis of tubular-shaped prophylactic. The method 1300 further includes rolling an open end of the prophylactic toward a closed end 1314. The method 1300 also includes capturing at least a portion of the first elongated tab and the second elongated tab in the rolled portion of the prophylactic 1316, and leaving a first free end of the first elongated tab outside the rolled portion 1318, and leaving a second free end of the second elongated tab outside the rolled portion 1320. The method 1300 also includes attaching the first free end of the first elongated tab to the second free end of the second elongated tab 1322. The method 1300 also includes deforming the rolled prophylactic to a non round shape 1324 and wrapping the first elongated tab to the second free end of the second elongated tab around the rolled end 1326, and inserting the deformed and rolled prophylactic into a cavity in a card package 1328.

A card package 100 includes a body. The body has an oblong cavity therein. The card package also includes a cover over the oblong cavity in the body, and a prophylactic within the oblong cavity. The prophylactic is packaged to fit within the oblong cavity in the body, and to maintain a shape capable of fitting within the body after removal from the cavity. In one example embodiment, the prophylactic has an oblong shape and in another example embodiment, the prophylactic has an hour-glass shape. In still another example embodiment, the body of the card package has a second oblong cavity therein. The card package also includes a second cover over the second oblong cavity, and a second prophylactic within the second oblong cavity. The second prophylactic is packaged to fit within the second cavity in the body, and to maintain a shape capable of fitting within the body after removal from the second cavity.

A card package 100 includes a body. The body has an oblong cavity therein. The card package also includes a cover over the oblong cavity in the body, and a prophylactic within the oblong cavity. The prophylactic further includes an elongated tubular member that has a closed end, and an open end. The prophylactic also includes a first elongated tab placed substantially parallel to the axis of the elongated tubular member, and a second elongated tab placed substantially parallel to the axis of the elongated tubular member. The open end of the tubular member is rolled toward the closed end wherein the first elongated tab has a length rolled into the rolled end, and a first free end, and wherein the second elongated tab has a length rolled into the rolled end, and a second free end. The second elongated tab has a length rolled into the rolled end, and a second free end. The first free end and the second free end are attached to one another to hold the rolled prophylactic device in a non round shape. In one embodiment, the first free end and the second free end include printing, such as printed instructions. In another embodiment, the first free end includes a first orientation indicator, and the second free end includes a second orientation indicator. In some embodiments, the first orientation indicator, and the second orientation indicator are viewable in low light conditions. The first orientation indicator, and the second orientation indicator are viewable when the prophylactic is in a proper orientation for application. In still other embodiments, the card package has a second oblong cavity in the body of the card package. The card package also includes a second cover over the second oblong cavity, and a second prophylactic within the second oblong cavity. In one embodiment, the cover and the second cover are joined to one another along a perforated line positioned over the body of the card package.

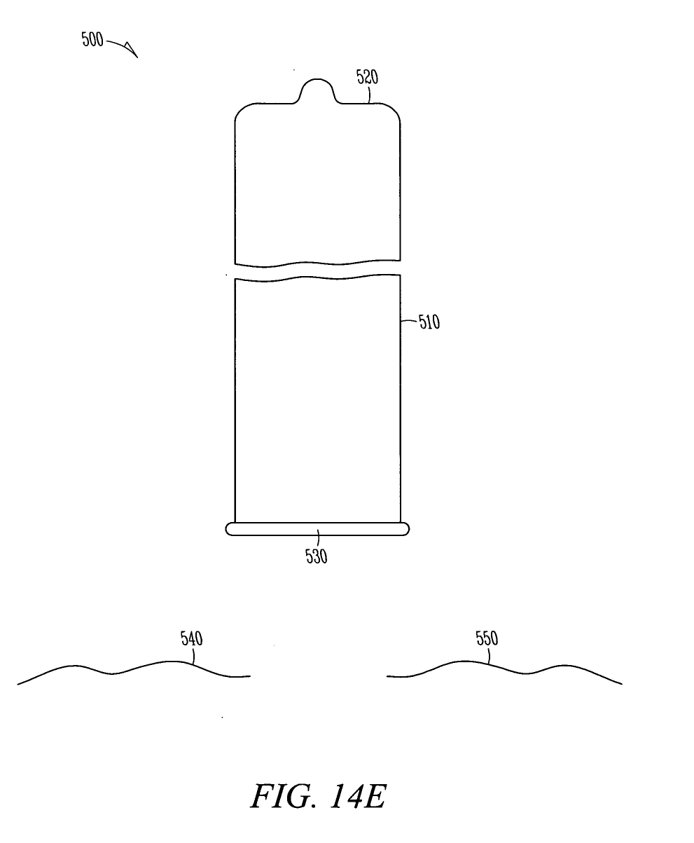

FIG. 14A is a cross sectional view along 14A-14A in FIG. 10, according to an example embodiment. FIGS. 14A-14E are also cross sectional views of various stages of deployment of the prophylactic device 500, according to an example embodiment. FIG. 14A shows the prophylactic device 500 having a closed end 520 and a rolled end 532 which is proximate the closed end. Rolled into the closed end 532 is a first tab 540 and a second tab 550. The first tab 540 includes a free end 542, and the second tab 550 includes a free end 552 which extend out beyond the rolled portion 532 of the prophylactic 500. To unroll the rolled portion 532, the free ends 542, 552 of the tabs 540, 550, respectively, are pulled downwardly as depicted by the arrows 1440 and 1450. As shown in FIGS. 14B, 14C and 14D, the free ends 552 and 542 continue to have a downwardly acting force 1440, 1450 or a force acting in the direction shown by the arrow depicted by 1440 and 1450. As the free ends 542, 552 of the tabs 540, 550 a force is continued to be applied to those tabs, the rolled end 532 continues to unroll thereby exposing more of the elongated tubular member 510 of the prophylactic. As shown in FIG. 14B, the elongated tubular member 510 is just beginning to be exposed. As the free ends 542, 552 continue to be pulled or forced in a direction depicted by arrows 1440, 1450, more of the elongated or tubular member 510 is exposed while the rolled end 532 continues to become smaller or continues to unroll. FIG. 14D shows the prophylactic 500 as rolled end 532 is almost completely unrolled. Near the end of the unrolling of the rolled end 532, the tabs 540, 550 are disengaged from the rolled end 532. As shown in FIG. 14E, the prophylactic 500 is fully deployed. The prophylactic 500 has a closed end, a main tubular member 510, and the opened end 532. As shown in FIG. 14E, the tabs 540 and 550 are completely disengaged from the prophylactic.

The foregoing description of the specific embodiments reveals the general nature of the invention sufficiently that others can, by applying current knowledge, readily modify and/or adapt it for various applications without departing from the generic concept, and therefore such adaptations and modifications are intended to be comprehended within the meaning and range of equivalents of the disclosed embodiments.

It is to be understood that the phraseology or terminology employed herein is for the purpose of description and not of limitation. Accordingly, the invention is intended to embrace all such alternatives, modifications, equivalents and variations as fall within the spirit and broad scope of the appended claims.

What is claimed is:

1. A prophylactic device comprising:
an elongated tubular member further comprising:
a closed end; and
an open end;
a first elongated tab placed substantially parallel to the axis of the elongated tubular member; and
a second elongated tab placed substantially parallel to the axis of the elongated tubular member, the open end of the tubular member rolled toward the closed end wherein the first elongated tab has a length rolled into the rolled end, and a first free end, and wherein the second elongated tab has a length rolled into the rolled end, and a second free end, the first free end and the second free end attached to one another to hold the rolled prophylactic device in a non round shape.

2. The prophylactic device of claim 1 wherein the non round shape is substantially elliptical.

3. The prophylactic device of claim 1 wherein the non round shape is substantially hour-glass shaped.

4. The prophylactic device of claim 1 wherein the first free end includes a first orientation indicator, and the second free end includes a second orientation indicator.

5. The prophylactic device of claim 4 wherein the first orientation indicator, and the second orientation indicator include printing.

6. The prophylactic device of claim 4 wherein the first orientation indicator, and the second orientation indicator include printed instructions.

7. The prophylactic device of claim 4 wherein the first orientation indicator, and the second orientation indicator are viewable in low light conditions.

8. The prophylactic device of claim 1 wherein the first elongated tab a first attached end, and the second elongated tab has a second attached end, the first attached end and the second attached end attached to elongated tubular member proximate the open end.

9. The prophylactic device of claim 1 wherein the first elongated tab a first other end, and the second elongated tab has a second other end, the first other end and the second other end being unattached to elongated tubular member.

10. A card package comprising:
a body, the body having an oblong cavity therein;
a cover over the oblong cavity in the body; and
a prophylactic within the oblong cavity, the prophylactic packaged to fit within the cavity in the body and to maintain a shape capable of fitting within the body after removal from the cavity.

11. The card package of claim 10 wherein the prophylactic has an oblong shape.

12. The card package of claim 10 wherein the prophylactic has an hour-glass shape.

13. The card package of claim 10 wherein the body has a second oblong cavity therein, the card package further including:
a second cover over the second oblong cavity; and
a second prophylactic within the second oblong cavity, the second prophylactic packaged to fit within the second cavity in the body and to maintain a shape capable of fitting within the body after removal from the second cavity.

14. A card package comprising:
a body, the body having an oblong cavity therein;
a cover over the oblong cavity in the body;
a prophylactic within the oblong cavity, the prophylactic further comprising
an elongated tubular member further comprising:
a closed end; and
an open end;
a first elongated tab placed substantially parallel to the axis of the elongated tubular member; and
a second elongated tab placed substantially parallel to the axis of the elongated tubular member, the open end of the tubular member rolled toward the closed end wherein the first elongated tab has a length rolled into the rolled end, and a first free end, and wherein the second elongated tab has a length rolled into the rolled end, and a second free end.

15. The card package of claim 14 wherein the second elongated tab has a length rolled into the rolled end, and a second free end, the first free end and the second free end attached to one another to hold the rolled prophylactic device in a non round shape.

16. The card package of claim 14 wherein the first free end and the second free end include printing.

17. The card package of claim 14 wherein the first free end and the second free end include printed instructions.

18. The card package of claim 14 wherein the first free end includes a first orientation indicator, and the second free end includes a second orientation indicator.

19. The card package of claim 18 wherein the first orientation indicator, and the second orientation indicator are viewable in low light conditions.

20. The card package of claim 18 wherein the first orientation indicator, and the second orientation indicator are viewable when the prophylactic is in a proper orientation for application.

21. The card package of claim 14 wherein the body has a second oblong cavity therein, the card package further including:
a second cover over the second oblong cavity; and
a second prophylactic within the second oblong cavity.

22. The card package of claim 21 wherein the cover and the second cover are joined to one another along a perforated line positioned over the body of the card package.

23. A prophylactic device comprising:
an elongated tubular member further comprising:
a closed end; and
an open end, the open end of the tubular member rolled toward the closed end; and
a first tab having a first free end near the closed end of the elongated tubular member; and
a second tab having a second free end near the closed end of the elongated tubular member, wherein the first free end and the second free end attached to one another to hold the rolled prophylactic device in a non round shape.

24. The prophylactic device of claim 23 wherein the first free end and the second free end include instructions.

25. The prophylactic device of claim 23 wherein the first free end and the second free end include an orientation indicator.

26. The prophylactic device of claim 23 wherein the first free end and the second free end include an orientation indicator viewable in low light conditions.

27. A method for assembling a card package that includes a prophylactic comprising:
placing a first elongated tab along a surface of a tubular-shaped prophylactic, the first elongated tab being substantially parallel to an axis of tubular-shaped prophylactic;
placing a second elongated tab along a surface of a tubular-shaped prophylactic, the second elongated tab being substantially parallel to an axis of tubular-shaped prophylactic;
rolling an open end of the prophylactic toward a closed end which further includes:
capturing at least a portion of the first elongated tab and the second elongated tab in the rolled portion of the prophylactic;
leaving a first free end of the first elongated tab outside the rolled portion; and
leaving a second free end of the second elongated tab outside the rolled portion; and
attaching the first free end of the first elongated tab to the second free end of the second elongated tab.

28. The method of claim 27 further comprising deforming the rolled prophylactic to a non round shape.
